(19) United States
(12) Patent Application Publication
(10) Pub. No.: US 2007/0196280 A1
(43) Pub. Date: Aug. 23, 2007

(54) In vivo magnetic resonance spectroscopy of aspartate transaminase activity

(75) Inventor: Jun Shen, North Potomac, MD (US)

Correspondence Address: KLARQUIST SPARKMAN, LLP, 121 S.W. SALMON STREET, SUITE #1600, PORTLAND, OR, 97204-2988, US

(73) Assignee: The Government of the U.S.A. as represented by the Sec. of the Dept. of Health and Human Services

(21) Appl. No.: 11/359,214

(22) Filed: Feb. 21, 2006

(51) Int. Cl. A61K 49/10

(52) U.S. Cl. 424/9.3

(57) ABSTRACT

A non-invasive method of assessing aspartate aminotransferase (AST) activity in vivo, comprising performing in vivo magnetization transfer spectroscopy on an organ of a living subject in which AST activity is to be measured, and determining a change in magnetic resonance signal intensity of reactants in an AST catalyzed reaction.

### FIELD

The methods disclosed herein relate to in vivo magnetic resonance spectroscopy (MRS) of enzyme activity.

### BACKGROUND

Aspartate transaminase (AST; also known as aspartate aminotransferase (AAT) or L-aspartate:α-ketoglutarate aminotransferase) is a ubiquitous enzyme that catalyzes the interconversion of aspartate (ASP) and α-ketoglutarate (α-KG) with glutamate (Glu) and oxaloacetate (OAA):
Asp+α-KGOAA+Glu
The activity of AST is known to change in a variety of pathological states, such as hepatitis, cholangitis, Gilbert's disease, cirrhosis, cardiac infarction, muscle dystrophy, leukemia, kidney inflammation and certain tumors. AST activity is therefore routinely assessed in plasma or serum blood tests as a non-invasive and inexpensive means to identify or follow the clinical condition of a patient. However, AST activity has not been routinely used to assess neurological diseases because the blood brain barrier prevents altered AST levels in the brain from being detectable in the serum. Intracerebral AST levels are known to change in a number of brain diseases, such as Huntington's disease and epilepsy, but this information could only be obtained by performing a brain biopsy of a patient or obtaining post-mortem tissue for analysis. Such invasive diagnostic techniques are clearly inappropriate for routine clinical use.

There is a significant medical need for the determination of AST activity in the brain and other organs. AST levels could be used to help diagnose or follow the progress of a variety of pathological states, such as hepatitis, cirrhosis, cardiac infarction, muscle dystrophy, kidney inflammation and various cancers. Serum AST levels are currently used in the clinical assessment of these conditions, but serum AST is a non-specific test that does not indicate the organ from which the aminotransferase originated. Moreover, most neurological conditions associated with altered AST activity can not be assessed at all with serum AST because the blood brain barrier prevents the brain AST from reaching the serum. A method that can explicitly link altered AST activity to a specific organ system on which the analysis is performed, without relying on indirect measurement of AST in serum or obtaining biopsy samples, would be very valuable.

In vivo nuclear magnetic resonance (NMR) spectroscopy has been used to detect rapid enzyme-catalyzed exchange reactions using phosphorus-31 magnetic resonance spectroscopy-based magnetization (saturation or inversion) transfer experiments. As a result, enzymology of creatine kinase, arginine kinase and ATP synthetase can be studied in vivo, where the magnetization transfer of the phosphate moiety was used as a tracer of energetic fluxes. However, most enzymatic reactions are too slow to be measured by magnetization transfer techniques, which require that the flux to be measured multiplied by T₁ of the observed metabolite constitutes a significant fraction of its pool size. The cerebral enzyme activity of AST measured in brain homogenate is high (70-130 μmol/g/min in brain (Erecinska et al., “Metabolism and role of glutamate in mammalian brain,” Prog Neurobiol. 35:245-296, 1990)). Both Glu and Asp in the AST reaction can be highly enriched using intravenous infusion of ¹³C-labeled substrates. However, the magnetization transfer effect of a reaction catalyzed by an enzyme with high activity Vₘₐₓ may or may not be measurable in vivo. It also depends on substrate availability versus the Michaelis-Menten parameter Kₘ. For example, the concentration of OAA is extremely low in vivo with a [substrate]/Kₘ ratio of <˜0.05. The Michaelis-Menten equation of the full AST transamination reaction predicts an in vivo transamination rate in the Glu+OAA→α-KG+Asp direction at <˜5% of its Vₘₐₓ with a magnetization transfer effect below the detection threshold of in vivo magnetic resonance spectroscopy.

In other words, in vivo substrate concentrations are usually much more dilute than Kₘ. The rate of the reaction of an enzyme in vivo is generally a very small fraction of Vₘₐₓ. Hence, in the absence of empirical evidence, it would be not possible to predict that the magnetization transfer effect of the aspartate aminotransferase reaction was measurable. This is particularly true considering that the in vivo concentration of oxaloacetate is extremely low (the lowest among all intermediates of energy metabolism).

### SUMMARY

According to a first embodiment, disclosed herein is a non-invasive method of assessing aspartate aminotransferase (AST) activity in vivo, comprising performing in vivo magnetization transfer spectroscopy on an organ of a living subject in which AST activity is to be measured, and determining a change in magnetic resonance signal intensity of reactants in an AST catalyzed reaction.

According to a second embodiment, disclosed herein is a method for detecting a physiological or pathological condition of an organ of a living subject, wherein a change in aspartate aminotransferase (AST) activity in the organ is indicative of the presence of a physiological or pathological condition. The method comprises performing in vivo magnetization transfer spectroscopy on an organ of a living subject in which AST activity is to be measured and determining a change in magnetic resonance signal intensity of reactants in an AST catalyzed reaction; and determining whether the measured AST activity indicates the presence of a physiological or pathological condition.

The foregoing and other objects, features, and advantages will become more apparent from the following detailed description, which proceeds with reference to the accompanying figures.

### BRIEF DESCRIPTION OF THE DRAWINGS

FIG. 1A is a comparison of a saturation transfer spectrum with the corresponding control spectrum for the half reaction α-KG [symbol] Glu using the 90° flip angle method described herein. TR=10000 ms. γBₗₛₐₜ=158 hz. lb=30. Upper trace: saturation transfer spectrum (NS=128); middle trace: control spectrum (NS=128); lower trace: difference spectrum.

FIG. 1B is a comparison of a saturation transfer spectrum with the corresponding control spectrum for the half-reaction α-KG [symbol] Glu after complete ischemia. When saturation transfer spectra (upper traces of FIGS. 1A and 1B) were acquired, continuous ¹³C RF saturation of the carbonyl carbon of α-KG at 206.0 ppm was employed. When control spectra were acquired (middle traces), the saturating pulse was placed at an equal spectral distance from Glu C2 but on the opposite side of α-KG C2. Glc: glucose; NAA: N-acetylaspartate. Lac: lactate. Intensity scale for upper and middle traces: ×1; for lower trace: ×4.

FIG. 2A shows the summed control and difference spectra with the continuous ¹³C RF saturation pulse centered on α-KG C2 at 206.0 ppm (n=11, γBₗₛₐₜ=158-628 Hz). Upper trace: summed control spectrum. Overlapping between Glu C2 at 55.7 ppm and Gln C2 at 55.2 ppm was minimized using the resolution-enhancing window function (lb=−30, gb=0.2). Intensity scale: ×1. Lower trace: corresponding difference spectrum. Intensity scale: ×4.

FIG. 2B shows the summed control and difference spectra with the continuous ¹³C RF saturation pulse centered on OAA C2 at 201.3 ppm (n=4, γBₗₛₐₜ=790 Hz). Upper trace: summed control spectrum (lb=−10, gb=0.03). Intensity scale: ×1. Lower trace: corresponding difference spectrum. Intensity scale: ×4.

FIG. 3 depicts a pulse sequence utilized in Example 2 as described in more detail below.

FIG. 4 shows a comparison of a saturation transfer spectrum with the corresponding control spectrum for the Glu→α-KG reaction when the carbonyl carbon of α-KG at 206.0 ppm was saturated.

FIG. 5 shows results for the carboxylic carbon region for Example 2 as described below in more detail, where 5a is the control spectrum without any pre-irradiation and 5b is the saturation transfer spectrum.

### DETAILED DESCRIPTION

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

For ease of understanding, the following terms used herein are described below in more detail:

Chemical Exchange: A physical process whereby nuclides initially bound to a first compound become bound to a second compound, and hence are physically transferred from the first to the second compound.

Chemical Exchange Dependent Saturation Transfer (CEDST): Refers to all saturation transfer processes between molecules that are dependent on chemical exchange between the molecules. An example of a CEDST process is described in more detail in U.S. Pat. No. 6,963,769, which is incorporated herein by reference in its entirety.

Non-invasive: In one aspect, “non-invasive” methods refer to methods that do not require transcranial introduction of an instrument. Non-invasive techniques can include the administration of a diagnostic substance intravenously or orally. Examples of invasive techniques include a tissue biopsy.

Organ: Refers to a defined biological structure that contains at least two different types of tissue functioning together for a common purpose. Examples of organs include brain, heart, lung, and skin.

Saturation Transfer (ST): “Saturation” refers to the destruction or randomization of the net magnetization in a sample using an applied magnetic field with or without spatial magnetic field gradients. Transfer refers to a physical process whereby this saturation is passed between different molecules by through-space interactions or direct chemical exchange from a first compound to a second compound. Saturation transfer is described below in more detail in connection with magnetization transfer.

Subject: Living multicellular vertebrate organisms, a category which includes human and veterinary subjects, for example mammals; farm animals such as pigs, horses, and cows; laboratory animals such as rodents and rabbits; birds, and primates.

The above term descriptions are provided solely to aid the reader, and should not be construed to have a scope less than that understood by a person of ordinary skill in the art or as limiting the scope of the appended claims.

Magnetization transfer effect is a type of NMR spectroscopy in which a biochemical marker of disease can be determined in living tissue. Magnetization transfer is accomplished by selectively introducing a non-equilibrium nuclear spin magnetization into an ensemble of nuclei which exchange with other unperturbed spin ensembles. If the exchange is rapid enough, the non-equilibrium spin magnetization can be observed to flow between the ensembles before it is relaxed by the T1 mechanism. In this manner, rate constants for chemical exchange are determined by observing the flow of an unusual type of nuclear label. An example of a magnetization transfer spectroscopy process is described in more detail in U.S. Pat. No. 5,050,609, which is incorporated herein by reference in its entirety.

In more detail, coupling between a NMR-invisible keto acid and the cognate NMR-detectable amino acid allows the spin state of the keto acid to influence the spin state of the amino acid through exchange processes catalyzed by transaminase. It is possible to saturate the keto acid spins preferentially using an off-resonance RF pulse. Such saturation is also referred to as magnetically labeling of the keto acid. The keto acid spins have different resonance frequencies from the amino acid spins. This saturation of the keto acid spins is transferred to the amino acid spins, depending upon the rate of exchange between the two types of molecules, and hence is detectable by MRS.

Disclosed herein are methods for using MRS to identify changes in AST activity and quantitate these changes. The aim is to provide the rate of the AST-catalyzed glutamate→α-ketoglutarate reaction of a given region of biological tissue. The method involves measuring a relative change in glutamate signal intensity. More particularly, disclosed herein are methods for determining the chemical reaction catalyzed by aspartate aminotransferase (AST) in living tissues using magnetization transfer or saturation transfer. The magnetization transfer or saturation transfer process employed herein may utilize a CEDST effect.

In one embodiment, a first carbon-13 magnetic resonance spectrum is obtained with signal enhancement by the nuclear Overhauser effect and proton decoupling (spectrum A). A second carbon-13 magnetic resonance spectrum is also obtained with simultaneous signal enhancement by the nuclear Overhauser effect and proton decoupling and saturation of the carbonyl magnetic resonance of α-ketoglutarate or oxaloacetate and proton decoupling (spectrum B). The nuclear Overhauser effect for spectrum A and the nuclear Overhauser effect for spectrum B are obtained with the same pulse sequence. Both spectrum A and spectrum B use the nuclear Overhauser effect and saturation except that in spectrum A, saturation is at carbonyl resonance frequency of α-ketoglutarate or oxaloacetate; in spectrum B, the saturation is placed at equal spectral distance but on the opposite side of carbonyl resonance frequency of α-ketoglutarate or oxaloacetate. The difference between these two spectrums is then determined to reveal the magnetization transfer effect. The inversion-recovery null (Tₗₙᵤₗₗ) of the detected amino acid α-carbon is measured with simultaneous saturation of corresponding carbonyl carbon resonance of its cognate keto acid, and the rate of the AST reaction is calculated with this information. The inversion-recovery null of the detected amino acid at the presence of saturation of its cognate keto acid was measured using an adiabatic three dimensionally localization method (Slotboom et al., “Adiabatic slice-selective rf pulses and a single-shot adiabatic localization pulse sequence,” Concept Magn Reson. 7:193-217, 1995). The nuclear Overhauser effect and proton decoupling and saturation of the cognate keto acid carbonyl carbon resonance was performed during Tₗₙᵤₗₗ measurement.

The methods disclosed herein involve the carbon magnetization transfer (CMT) effect and in vivo detection of the CMT effects of the α-ketoglutarate [symbol] glutamate and the oxaloacetate [symbol] aspartate reactions, both of which are catalyzed by aspartate aminotransferase. In one example, by saturating the carbonyl carbon of α-ketoglutarate at 206 ppm in α-chloralose anesthetized adult rat brain, the unidirectional glutamate→α-ketoglutarate flux was determined to be 78±9 μmol/g/min (mean±SD, n=11) following intravenous infusion of [1,6-¹³C₂]D-glucose. Because of the large chemical shift separation between the α-carbons of the amino acids and the carbonyl carbons of the corresponding cognate keto acids, the spillover of the saturation radiofrequency pulses to the α-carbon resonances was negligible. The magnetization transfer effects of aspartate aminotransferase-catalyzed reactions can be used as new biomarkers accessible to non-invasive in vivo magnetic resonance spectroscopy techniques.

In the methods disclosed herein it is the rate of the dynamic reaction that is measured (in contrast to the concentration of glutamate or the concentration of the AST enzyme per se). A concentration change (e.g., N-acetyl aspartate or glutamate) could be due to the results of many different enzyme reactions. Measuring a certain reaction provides much more specific information. The change in the rate of a single reaction may or may not change the concentration and vice versa. In the specific case of AST, if the AST reaction rate changes, the concentration of glutamate may not change. But the signal in the difference spectrum due to magnetization transfer effect will change.

Various possible RF pulse sequences can be used to generate the MRS data. One embodiment involves direct ¹³C-detected MRS. A second embodiment involves proton detection of carbon to proton coherence transfer methods. A third embodiment involves direct proton detection.

Direct ¹³C-detected MRS can utilize surface coil localization and/or volume-selection using RF pulses. An example of a surface coil localization pulse sequence for direct ¹³C-detected MRS is set forth in the Examples section below. Volume selection methods using RF pulses such as PRESS, STEAM, and ISIS can be used to localize a tissue volume for measuring the AST reactions. In the Examples section below, an adiabatic version of PRESS was used to measure the inversion-recovery null of Glu C2 (the second carbon position of Glu) at 55.7 ppm while saturating the carbonyl carbon of α-KG from a 9-mm×5-mm×9-mm (405 μL) voxel using a 4-ms hyperbolic secant inversion pulse (phase factor=5, 1% truncation) followed by direct, single-shot and adiabatic, 3D localization of ¹³C-labeled spins. The adiabatic PRESS was initially described by Slotboom and Bovee (Slotboom et al., “Adiabatic slice-selective rf pulses and a single-shot adiabatic localization pulse sequence,” Concept Magn Reson. 7:193-217, 1995).

Proton detection of carbon to proton coherence transfer methods include saturation of the carbonyl carbon of α-ketoglutarate at 206.0 ppm and nuclear Overhauser enhancement of the α-carbon of glutamate that is the same as described in connection with the Examples section below. There are three efficient ways to transfer the thermal equilibrium magnetization of ¹³C to proton using the one-bond coupling between ¹³C and its proton(s) ¹J_{CH}: INEPT, DEPT and heteronuclear Hartmann-Hahn transfer, all of which can be used to measure magnetization transfer effect catalyzed by AST.

Carbon to proton coherence transfer using INEPT. The INEPT method for proton detection of the chemical exchange is based on a single-shot, mostly adiabatic, proton-localized INEPT-based ¹H→¹³C magnetization transfer pulse sequence (Li et al., “In vivo single-shot proton-localized ¹³C MRS of rhesus monkey brain,” NMR Biomed 18:560-569, 2005). The method uses a i) 1-ms adiabatic half-passage pulse with an end phase along the x axis of RF rotating frame for nonselective excitation of ¹³C equilibrium signals; ii) a pair of identical hyperbolic secant pulses (1.5 ms, μ=4, 1% truncation) to generate an adiabatic double spin echo; iii) a 1-ms hyperbolic secant pulse applied to the proton channel to reintroduce heteronuclear J evolution; iv) after a delay of ¼¹J_{CH}, a 1-ms time-reversed adiabatic half-passage pulse with a starting phase along the y axis of RF rotating frame to convert the antiphase 2¹H_{z}¹³Cₓ term into longitudinal two-spin order 2¹H_{z}¹³C_{z}; v) immediately after creation of 2¹H_{z}¹³C_{z}, outer volume suppression and water suppression to remove artifacts; vi) the single-shot, fully adiabatic PRESS sequence (Slotboom et al., “Adiabatic slice-selective rf pulses and a single-shot adiabatic localization pulse sequence,” Concept Magn Reson. 7:193-217, 1995) to convert 2¹H_{z}¹³C_{z} into 2¹H_{y}¹³C_{z}; vii) a final sech pulse (1.5 ms, μ=4, 1% truncation) applied to the 13C channel to reintroduce heteronuclear J evolution; viii) ¹³C CW decoupling after a delay of ¼¹J_{CH} with decoupler frequency centered at the α-carbon resonance of the observed amino acid.

Carbon to proton coherence transfer using DEPT. DEPT is less affected by B₁ inhomogeneity because it uses less RF pulses. The DEPT sequence per se uses i) a 1-ms ¹³C adiabatic half-passage pulse with its end phase on x; ii) a pair of identical 13C hyperbolic secant pulses to produce a pure phase adiabatic spin echo; iii) At ½¹J_{CH} before ¹³C spin echo center, a 1-ms proton adiabatic half-passage pulse (the 90° excitation pulse of adiabatic PRESS) to generate 2¹Hₓ¹³C_{y}; iii) at the ¹³C spin echo center, a 1-ms time-reversed adiabatic half-passage pulse with its start phase on y is to generate 2¹Hₓ¹³C_{z}; iv) the rest of the adiabatic PRESS sequence for proton spatial localization and rephasing of the antiphase 2¹Hₓ¹³C_{z} into H_{y}; v) ¹³C CW decoupling with decoupler frequency centered at the α-carbon resonance of the observed amino acid.

Carbon to proton Hartmann-Hahn coherence transfer. Carbon to proton Hartmann-Hahn coherence transfer uses i) a 1-ms ¹³C adiabatic half-passage pulse, ii) CW spin-lock pulses satisfying the Hartmann-Hahn condition: γ_{H}B_{1H}=γ_{C}B_{1C}; iii) adiabatic PRESS without its proton adiabatic half-passage pulse; iv) ¹³C decoupling with decoupler frequency centered at the α-carbon resonance of the observed amino acid.

The direct proton detection method uses PRESS, STEAM or ISIS localization or chemical shift imaging with incorporation of a CW pulse for saturation at the resonance frequency of α-ketoglutarate H3 at 3.0 ppm.

As mentioned above, a first carbon-13 magnetic resonance spectrum is obtained with signal enhancement by the nuclear Overhauser effect and proton decoupling (spectrum A). The nuclear Overhauser effect is used to enhance the detection sensitivity. In general, the nuclear Overhauser effect is measured between an α-proton of an amino acid such as glutamate and an α-carbon of an amino acid such as glutamate. Broadband ¹H→¹³C nuclear Overhauser enhancement can be generated by a variety of pulse sequence(s). The general requirement of the pulse sequence(s) is to maintain the proton magnetization at or close to zero. In one example, the nuclear Overhauser enhancement can be generated with a train of non-selective hard pulses with a nominal flip angle of 180° spaced at 100 ms apart.

The proton decoupling involves the decoupling of the protons attached to glutamate, particularly the α-proton of glutamate. Since the proton decoupling is broadband, the other protons are also decoupled simultaneously with the decoupling of the α-proton of glutamate. The proton decoupling can be generated by a variety of pulse sequence(s). The general requirement of the pulse sequence(s) is to remove spin evolution due to scalar coupling. In one example, the proton decoupling can be achieved with a WALTZ-4 decoupling scheme based on a 400-μs nominal 90° rectangular pulse.

The nuclear Overhauser enhancement is generated first, and then proton decoupling is generated during acquisition of NMR signals.

As described above, a second carbon-13 magnetic resonance spectrum is also obtained with simultaneous (i.e., time-shared) signal enhancement by the nuclear Overhauser effect and saturation of the carbonyl magnetic resonance of α-ketoglutarate or oxaloacetate and proton decoupling (spectrum B). In other words, the saturation pulse on carbonyl resonance frequency of α-ketoglutarate or oxaloacetate and the nuclear Overhauser effect pulse on glutamate or aspartate are executed simultaneously by leaving a train of 0.1 ms gap in the keto acid saturation pulse to insert a train of 180 degree pulses to invert glutamate for generation of the nuclear Overhauser effect. The nuclear Overhauser effect and proton decoupling are generated as described above. The saturation of the carbonyl magnetic resonance of α-ketoglutarate or oxaloacetate can be accomplished by a variety of pulse sequence(s). In one example, when saturation transfer spectra were acquired, ¹³C RF saturation of the carbonyl carbon of α-KG at 206.0 ppm or the carbonyl carbon of OAA at 201.3 ppm was performed. The saturation could be performed on other carbons instead of the carbonyl carbon. For instance, the saturation could be performed on C1 (at 182.2 ppm) and C3 (at 31.4 ppm) of α-KG, and C1 (at 169.3 ppm) and C3 9 at 49.9 ppm) of OAA. Saturation of the carbonyl carbon is the most convenient because it is far away from the corresponding α-carbon of glutamate or aspartate.

The control spectra may be obtained by using the same pulse sequence and parameters as described above except that the saturation pulse was placed at an equal spectral distance from the observed amino acid signal but on the opposite side of its cognate keto acid signal.

The difference spectra can be obtained by known methods. For example, data can be first zerofilled to 16 K. Then at least one window function can be applied (Three window functions were used in the Example set forth below: lb=30; lb=−30, gb=0.2; lb=−10, gb=0.03) for either resolution or sensitivity enhancement. The control spectrum is phased using both zero order and first order phase corrections. Then the same phase parameters are applied to the saturation spectrum. The saturation spectrum is then subtracted point-by-point from the control spectrum to obtain the difference spectrum.

Acquiring the NMR data described above may also involve administering at least one diagnostic substance to the subject prior to subjecting the subject to the MR pulses described above. Such diagnostic substances may be liquids that can be administered by any number of techniques (e.g., intravenously via direct injection or via an IV line, oral ingestion, etc.). In the embodiments involving direct ¹³C methods or the methods using ¹³C to proton coherence transfer (INEPT, DEPT and Hartmann-Hahn transfers), a ¹³C isotope may be administered. For example, a [1-,¹³C]-D-glucose- or [1,6-,¹³C₂]-D-glucose-containing composition (at 50-99% ¹³C fractional enrichment) may be used for administering a ¹³C isotope. The composition may be administered at 1M in saline for intravenous or direct injection or at 1M in water for oral administration.

The methods disclosed herein can be targeted to be organ-selective. In other words, a predetermined section or volume of an organ may be selected for irradiation in order to determine the AST activity in that section or volume of the organ. The techniques for targeting a certain section or volume of an organ for MR spectroscopy in general are well known in the art. Such organ-selective information is much more specific compared to the conventional non-specific serum AST test that does not indicate the organ from which the aminotransferase originated. For example, one could select an organ with suspected pathology to measure the AST activity.

As mentioned above, changes in AST activity are specifically associated with certain normal and abnormal conditions and diseases, and the in vivo methods disclosed herein are useful for identification, targeting, diagnosis, and the like of such conditions and diseases. A “condition” may be physiological (e.g., phenotype, genotype, fasting, water load, exercise, hormonal cycles, etc.) or pathological. Included among conditions and diseases is the degree of a condition or disease, for example, the progress of phase of a disease. The degree may be temporal and/or severity. The methods also can be used for monitoring the effectiveness of, and/or patient compliance with, a therapy. Further applications of the methods include prognosis (e.g., predicting the future outcome) and risk assessment to identify people at risk of suffering from a particular condition.

The methods described herein are particularly suitable for detecting neurological conditions such as Huntington's disease, olivopontocerebellar atrophy, epilepsy, and schizophrenia. The methods described herein can also be used to detect other conditions such as, for example, hepatitis, cholangitis, Gilbert's disease, cirrhosis, cardiac infarction, muscle dystrophy, leukemia, kidney inflammation, certain tumors, and other conditions in which AST activity is a biomarker. For example, in vivo MRS spectra may be collected and evaluated as described above to determine AST activity for various states of a subject (e.g., healthy (control) subjects and diseased subjects). The in vivo MRS spectra library then can be consulted to evaluate whether or not a particular condition is present in a subject. For example, based on brain samples obtained from therapeutic neurosurgery, AST activity is significantly different between spiking and nonspiking brain tissues. One could measure AST activity using MRS from a corresponding contralateral brain region and compare it to the suspected seizure focus. In another approach one can measure AST activity from a specific region in the brain (e.g., temporal lobe) from a number of age- and sex-matched healthy subjects and compare it to that of a patient.

According to another aspect of the methods disclosed herein, AST in the blood can be measured to detect necrosis and tissue damage due to diseases of the heart, liver, muscle, or biliary tract.

The methods described herein can be used to monitor the effectiveness of a therapy in which AST activity is an indicator. This method would include determining, prior to therapy, a baseline AST activity via the MRS methods described above, administering a therapy to the subject, and detecting any change in the AST activity via MRS in the subject compared to the baseline AST activity, wherein the change in the AST activity is indicative of the effectiveness of the therapy. Examples of therapies in which AST activity is, or could be, an indicator of the effectiveness of the therapy include administration of taxol to treat breast cancer, gabapentin or topiramate to treat epileptic patients and haloperidol to treat schizophrenic patients.

The MR spectroscopy methods describe herein can be implemented using any MRS and/or MRI apparatus and systems such as those operating at field strengths at or above 1.5 Tesla. MRS and/or MRI apparatus and systems are available from Bruker Medical GmBH, Caprius, Esoate Biomedica, Fonar, GE Medical Systems, Hitachi Medical Systems America, Intermagnetics General Corporation, Lunar Corp., Magne Vu, Marconi Medicals, Philips Medical Systems, Shimadzu, Siemens, and Toshiba America Medical Systems. Apparatus and systems having heteronuclear capability (e.g., having multichannel and broadband) are especially suitable. Those skilled in the art will be aware of variations which may be required to adapt the procedure for other equipment.

### EXAMPLE 1

Animal Preparation

All experiments were performed using an 11.7 Tesla 89-mm bore vertical magnet (Bruker Biospin, Billerica, Mass.). A home-built transmit/receive concentric surface ¹³C (circular, diameter: 10 mm)/¹H (octagonal, diagonal: 25 mm) radiofrequency (RF) coil system was used to minimize extracranial contributions. The homebuilt RF coil system is described in Li et al., “Integrated RF probe for in vivo multinuclear spectroscopy and functional imaging of rat brain using an 11.7 Tesla 89-mm bore vertical microimager,” MAGMA 18:119-127, 2005 (which article is incorporated herein by reference in its entirety), except that the homebuilt RF coil system included an inner coil tuned to ¹³C resonant frequencies and outer coil tuned to proton resonant frequencies.

Male Sprague-Dawley rats (185±17 g, no. of rats used=5) fasted for 24 hours with free access to drinking water were studied to measure the CMT effects in the rat brain as approved by the National Institute of Mental Health (NIMH) Animal Care and Use Committee. On three rats, the measurement of the α-KG [symbol] Glu CMT effect was repeated several times with three different power settings for irradiation of the carbonyl carbon of α-KG. The rats were orally intubated and ventilated with a mixture of 70% N₂O/30% O₂ and 1.5% isoflurane. The left femoral artery was cannulated for measuring arterial blood gases (pO₂, pCO₂), pH, plasma glucose concentration using a blood analyzer (Bayer Rapidlab 860, East Walpole, Mass.), and for monitoring arterial blood pressure. End-tidal CO₂, tidal pressure of ventilation, and heart rate were also monitored. Two femoral veins were also cannulated, one for intravenous infusion of α-chloralose (initial dose: 80 mg/kg supplemented with a constant infusion of 26.7 mg/kg/h throughout the experiment), the other for intravenous infusion of [1,6-¹³C₂]glucose (1-¹³C, fractional enrichment: 0.99; 6-¹³C, fractional enrichment: 0.97, Cambridge Isotope Labs, Andover, Mass.). The scalp was removed to minimize subcutaneous lipid contamination of in vivo ¹³C data. After surgical preparation, isoflurane was discontinued and anesthesia was switched to α-chloralose with pancuronium bromide administrated (2-3 mg/kg, i.v.) to maintain immobilization. Rectal temperature was maintained at 37.5±0.5° C. using an external pump for water circulation (BayVoltex, Modesto, Calif.). Intravenous infusion of [1,6-¹³C₂]glucose was started approximately 1 hour prior to ¹³C data acquisition. The infusion protocol consists of an initial bolus of 162 mg/kg/min of 1.1 M [1,6-¹³C₂]glucose in the first five minutes followed by constant-rate infusion of the same glucose solution. The total plasma glucose level was sampled every 30 minutes and maintained approximately constant at an average concentration of 20.9 mM. Normal arterial plasma physiological parameters were maintained except that the plasma glucose concentration reached hyperglycemic levels. For postmortem measurements, 1.5 mL 3 M KCl was injected into the femoral vein prior to spectroscopy data acquisition.

By way of background, for in vivo spectroscopy studies of brain metabolism and neurotransmission, rats are preferred in many occasions because of their much larger brain size which are well-characterized and are genetically similar to humans. In vivo NMR studies of rat brains typically are accurately predictive of the efficacy of similar in vivo NMR techniques in human brains because rat and human AST proteins share the same ancestral gene. For example, the human mitochondrial aspartate aminotransferase is 95% similar to the rat one (Christen et al., Transaminase, John Wiley & Sons, New York, 1985; Pol et al., “Nucleotide sequence and tissue distribution of the human mitochondrial aspartate aminotransferase mRNA,” Biochem Biophys Res Commun. 157(3):1309-15, 1988). A surgical dose of α-chloralose preserves the functional-metabolic coupling in the somatosensory cortex of rat brains while physiological parameters can be maintained by adjusting the rate and volume of ventilation based on periodic blood gas sampling.

In Vivo NMR Spectroscopy

Either a 90° excitation or a 45° excitation, surface-coil-localized, interleaved acquisition method was used to measure the CMT effects. With the 90° excitation method, a 1-ms adiabatic half-passage pulse was used for non-selective 90° ¹³C excitation. TR=10000 ms. ¹H decoupling was achieved with the use of a WALTZ-4 decoupling scheme based on a 400 μs nominal 90° rectangular pulse. The decoupling pulse train was executed for ˜100 ms at the start of data acquisition. Broadband ¹H→¹³C nuclear Overhauser enhancement was generated using a train of non-selective hard pulses with a nominal flip angle of 180° spaced at 100 ms apart. When saturation transfer spectra were acquired, ¹³C RF saturation of the carbonyl carbon of α-KG at 206.0 ppm or the carbonyl carbon of OAA at 201.3 ppm was performed. When control spectra were acquired, the saturating pulse was placed at an equal spectral distance from the observed spin but on the opposite site of the saturated spin. The saturated and control spectra were interleaved every FID. With the 45° excitation method, a nominal 45° rectangular pulse was used for ¹³C excitation. TR=4100 ms. The saturation transfer and the control spectra were interleaved every eight FIDs. Preceding each eight-FID block, two dummy scans were used. Data were zerofilled to 16 K. The ¹³C signals in the 53-56 ppm region were analyzed using the MATLAB curve-fitting toolbox (The MathWorks, Inc., Natick, Mass.) in the frequency domain. The inversion-recovery null of Glu C2 (the second carbon position of Glu) at 55.7 ppm while saturating the carbonyl carbon of α-KG was measured from a 9-mm×5-mm×9-mm (405 μL) voxel using a 4-ms hyperbolic secant inversion pulse (phase factor=5, 1% truncation) followed by direct, single-shot and adiabatic, 3D localization of ¹³C-labeled spins. TR/TE=11000/18 ms. The parameters for saturation of α-KG, generation of ¹H→¹³C nuclear Overhauser enhancement and ¹H decoupling used in the T₁ null measurement were the same as described above. The ¹³C carrier frequency was centered at Glu C2.

Results

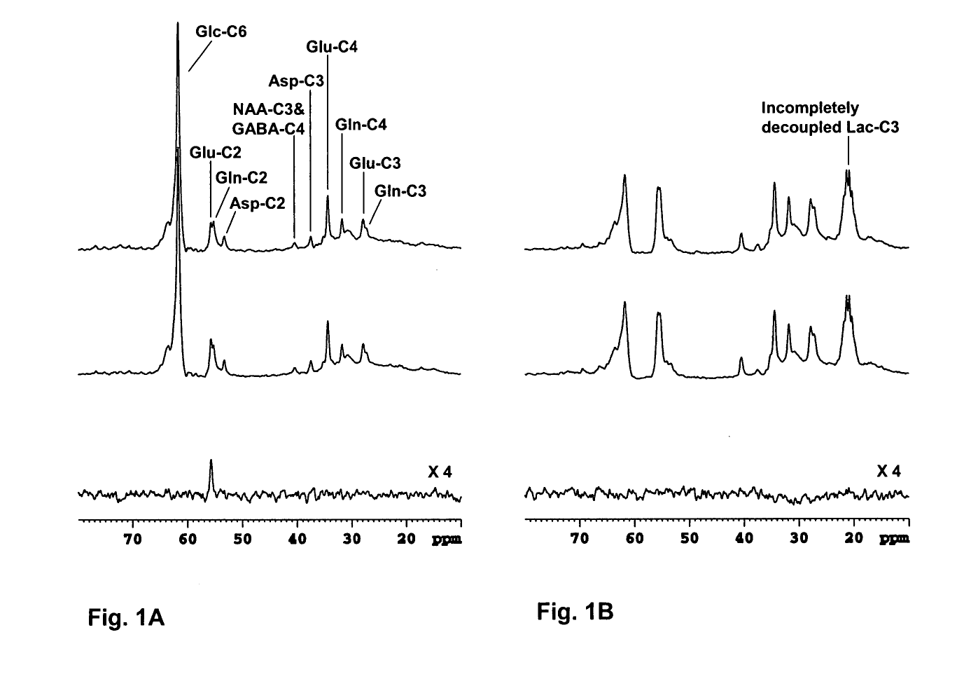

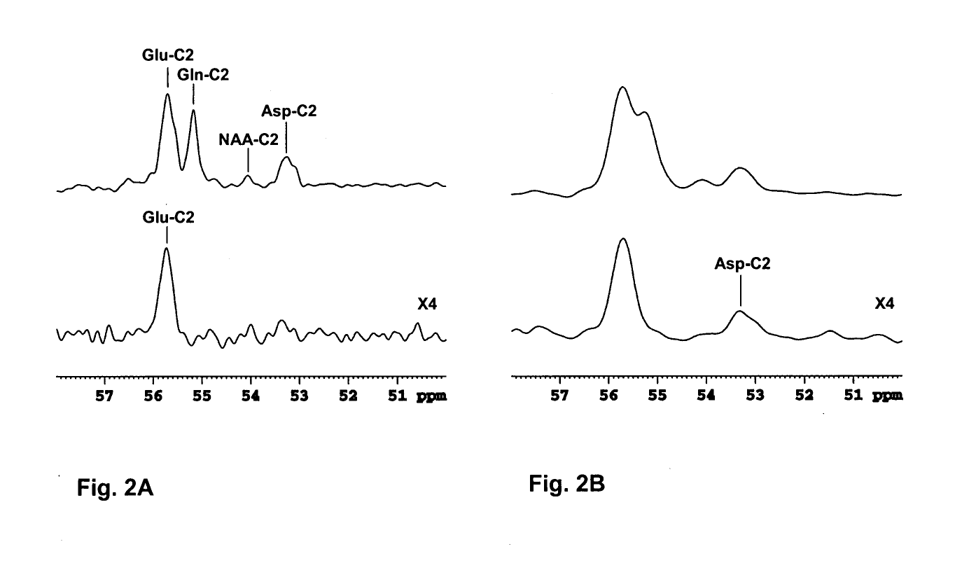

FIG. 1A shows a comparison of a saturation transfer spectrum with the corresponding control spectrum for the half-reaction α-KG Glu using the 90° excitation method. Nominal γBₗₛₐₜ=158 Hz (γ is the gyromagnetic ratio). The ¹³C RF saturation pulse was centered on α-KG C2. No. of scans (NS)=128 with 30 Hz exponential line broadening. Before the end of the study, the saturation transfer experiment was repeated immediately after intravenous injection of KCl. The postmortem results are shown in FIG. 1B. All experimental and processing parameters in FIG. 1B were kept the same as those in FIG. 1A except that γBₗₛₐₜ=628 Hz. The lactate (Lac) C3 peak at 20.8 ppm in FIG. 1B was not completely decoupled because the proton decoupler frequency was centered at 3.5 ppm. The difference spectra showed significant intensity change in Glu C2 at 55.7 ppm in live rats. The ratio of the intensity of Glu in the difference spectrum to that of the deconvoluted Glu signal in the control spectrum (ΔM/M₀)_{Glu} was determined to be 24±2% at γBₗₛₐₜ=158 Hz (mean±SD, n=4). The completeness of RF saturation of α-KG was tested by increasing γBₗₛₐₜ to 315 and to 628 Hz. No significant differences among the (ΔM/M₀)_{Glu} values determined using the three different γBₗₛₐₜ settings were found. The (ΔM/M₀)_{Glu} values were lumped together and averaged to be 24±2% (mean±SD, n=11). When the (ΔM/M₀)_{Glu} value(s) from each rat were averaged and then the means from different rats were averaged for cross-animal analysis, essentially the same mean and SD were obtained. The overlap between Glu C2 and Gln C2 can be minimized by the use of a resolution-enhancing window function (lb (exponential broadening factor)=−30, gb (Gaussian broadening factor)=0.2). The summed control spectrum and the corresponding summed difference spectrum processed using the aforementioned resolution-enhancing window function are shown in FIG. 2A. The T₁ null time (TI) of Glu C2 with the saturation of α-KG was determined to be 1.3±0.1 s (mean±SD, n=5). The full inversion of the spins inside the 405 μL voxel by the 4-ms T₁ null pulse was verified experimentally using a phantom sample which loads approximately the same as a rat head. The possible contribution from the non-specific off-resonance magnetization transfer effect presumably due to a small, immobilized glutamate pool was investigated by shifting the frequency of the saturation pulse from α-KG C2 to ±100 kHz while keeping the frequency of the continuous irradiation pulse in the control scans unchanged (i.e., at equal spectral distance from Glu C2 but on the opposite side of α-KG; same as in FIGS. 1 and 2A). No non-specific off-resonance ¹³C magnetization transfer effect was detected for glucose, glutamate or other amino acids in the difference spectra using the 90° excitation method with TR=10000 ms, NS=128×2, γBₗₛₐₜ=315 or 628 Hz. The ratio of the intensity of Asp in the difference spectrum to that of the Asp signal in the control spectrum (ΔM/M₀)_{Asp} was determined from spectra acquired with the saturation pulse (γBₗₛₐₜ=790 Hz) centered at the frequency of the carbonyl carbon of OAA. The 45° excitation method was used. TR=4100 ms. NS=384. (ΔM/M₀)_{Asp} was determined to be 27±3% (mean±SD, n=4). FIG. 2B shows the CMT effect when the saturation pulse was centered at the frequency of the carbonyl carbon of OAA. The summed control and difference spectra were plotted in FIG. 2B. Because of the relatively strong saturation pulse used to irradiate OAA C2 at 201.3 ppm, significant CMT effect due to the α-KG [symbol] Glu half-reaction was also seen in FIG. 2B.

Using the simple two-site exchange model described by the classical Bloch-McConnell equations, the pseudo first-order rate constant k is given by
k=(ΔM/M₀)/Tₗₛₐₜ [1]
where Tₗₛₐₜ is the apparent T₁ of an amino acid during saturation of its cognate keto acid. Using Eq. [1], Tₗₛₜₐ=TI/ln2, and V_{Glu→\tilde{α}KG}=k_{Glu→\tilde{α}KG}[Glu], the pseudo first-order rate constant and the corresponding unidirectional Glu→\tilde{α}KG flux in α-chloralose anesthetized rat brain were calculated to be 0.13±0.01 s⁻¹ and 78±9 μmol/g/min (mean±SD, n=11), respectively. [Glu] was set to be 10.2 μmol/g based on analysis of brain perchloric acid extracts.

There are also other aminotransferase reactions (e.g., alanine aminotransferase, GABA transaminase, branched-chain amino acid aminotransferase, ornithine aminotransferase and glutamine aminotransferase) which involve the α-KG [symbol] Glu half-reaction. Of them, only alanine aminotransferase, GABA transaminase, and ornithine aminotransferase use α-KG as the primary amino group receptor. In brain, the aminotransferase activities using the α-KG [symbol] Glu half-reaction are overwhelmingly dominated by that of AST, which represents >97% of the Glu-related aminotransferase activities (Erecinska et al., “Metabolism and role of glutamate in mammalian brain,” Prog Neurobiol. 35:245-296, 1990). Glutamate dehydrogenase also involves the α-KG [symbol] Glu exchange, but in brain homogenate, its activity measured in the direction of reductive amination is less than the activity of AST (70-130 μmol/g/min measured in both directions) by a factor of 10-20. The Vₘₐₓ of glutamate dehydrogenase in the direction of oxidative deamination (Glu→α-KG, measured in this study) is approximately 1/10 of its Vₘₐₓ in the opposite direction.

Mechanistically, AST catalyzes an enzyme-substitution, or “ping-pong” reaction in that Glu (originated from its cognate α-KG) is released from the pyridoxal-enzyme-glutamate complex before the binding of Asp required for the full AST reaction. After the binding of Asp, its amino group can be transferred to regenerate the pyridoxamine-enzyme complex for the next α-KG. Ping-pong reaction mechanisms usually involve partial reactions. That is, the exchange between Glu and α-KG catalyzed by AST does not need the presence of OAA or Asp and vice versa. The partial reactions α-KG [symbol] Glu and OAA [symbol] Asp lead to rapid exchange between the amino acids and their cognate keto acids. Simultaneously, AST also catalyzes the exchange of Glu H2 and Asp H2 with water or deuterium. The unidirectional Glu→α-KG flux measured here includes both the “futile” exchange between α-KG and Glu, and the full AST transamination reaction which involves transfer of amino groups between Glu and Asp. The presence of the futile exchange allows ¹³C isotope exchange between Glu and α-ketoglutarate to proceed at a rate much faster than that of the full transamination per se. It is most likely that the measured Glu→α-KG flux rate is dominated by the α-KG [symbol] Glu partial reaction, considering the very low [substrate]/Kₘ ratio of OAA. In fact, the unidirectional Glu→α-KG flux rate determined in vivo is close to the reported enzyme activity value of AST-catalyzed full transamination reaction in the Glu+OAA→α-KG+Asp direction (130 μmol/g/min), although AST is far from being saturated in vivo for this reaction. Metabolic compartmentation, enzyme-enzyme associations, substrate binding, and the near-open in vivo metabolic system also affect the AST reactions, which cannot be accurately characterized by the simple Michaelis-Menten equation for the enzyme-substitution mechanism governing the kinetics of the full AAT reaction in vitro.

The contribution to the measured unidirectional Glu→α-KG flux from the reaction catalyzed by glutamate dehydrogenase, which is a mitochondrial enzyme in brain, should be very small. While glutamate is located predominantly in cytoplasm of glutamatergic neurons, there is a substantial amount of evidence suggesting that glutamate dehydrogenase is located predominantly in a separate compartment, the astrocytes. Nevertheless, glutamate dehydrogenase activity in neurons has been found to be of significance. The glutamate dehydrogenase activity in the direction of reductive amination in the mitochondrial subfraction SM2 from cortical synaptic terminals has been shown to be comparable to the activity of AST in the same subfraction. While glutamate dehydrogenase is localized in mitochondria, AST is in both mitochondria and cytoplasm. The CMT effect determined here was originated from both cytoplasm and mitochondria including the SM2 subfraction from synaptic terminals. A relatively significant contribution from glutamate dehydrogenase in SM2 of cortical synaptic terminals should not add significantly to the likelihood of a significant glutamate dehydrogenase contribution to the global CMT effect on Glu, bearing in mind that the brain glutamate dehydrogenase activity in the Glu→α-KG direction (oxidative deamination) is approximately <1% (˜1:20-1:10× 1/10) of that of AST. Unlike AST, glutamate dehydrogenase does not catalyze a partial reaction similar to the futile exchange between α-KG and Glu catalyzed by AST. Therefore, significant contributions from other reactions including the glutamate dehydrogenase reaction to the Glu→α-KG flux measured in this example are unlikely. The measured Glu→α-KG flux should be overwhelmingly dominated by the flux facilitated via AST.

As shown in FIGS. 1A and 2, there are no significant changes in the signal intensities of the spectrally resolved Gln C2 at 55.2 ppm and the N-acetylaspartate C2 at 54.1 ppm upon saturating the α-keto acids. Only the intensity of Glu C2 at 55.7 ppm and that of Asp C2 at 53.2 ppm were affected as expected. The concentrations of α-KG and OAA are known to fall dramatically and immediately after death as a result of complete blockade of oxidative reactions due to complete ischemia (Siesjo, Brain energy metabolism, John Wiley & Sons, Chichester, 1978). Acquisition of the spectra shown in FIG. 1B was initiated immediately after cardiac arrest by lethal injection of KCl. As shown in FIG. 1B, the CMT effect was no longer measurable due to postmortem depletion of α-KG and OAA. The absence of any signal in the difference spectrum of the postmortem brain indicates that the AST reaction is terminated due to the postmortem depletion of α-KG and OAA.

The in vivo concentration of α-KG was reported to be 0.136 μmol/g (Siesjo, Brain energy metabolism” John Wiley & Sons, Chichester, 1978). At a flux rate of 78 μmol/g/min, the turnover time of α-KG is approximately 0.1 s assuming a single metabolic compartment. A full mathematical description of the two-site exchange model was proposed recently (Zhou et al., “Quantitative description of proton exchange processes between water and endogenous and exogenous agents for WEX, CEST, and APT experiments,” Magn Reson Med. 51:945-952, 2004) to analyze errors in saturation transfer due to incomplete RF saturation, which takes into account of transverse magnetization-related exchange terms. The steady state longitudinal magnetization of Glu C2 under our experimental conditions is described by Eq. [32] in Zhou et al., “Quantitative description of proton exchange processes between water and endogenous and exogenous agents for WEX, CEST, and APT experiments,” Magn Reson Med. 51:945-952, 2004. The following parameter values were either taken from literature, or determined in vivo, or estimated based on in vitro measurements: [Glu]=10.2 μmol/g; [α-KG]=0.136 μmol/g (Siesjo, Brain energy metabolism, John Wiley & Sons, Chichester, 1978). Since AST has specificity for the keto form, which is the dominant form of α-KG at neutral pH, the concentration of α-KG was not corrected for its minor gem-diol content.); k_{Glu→\tilde{α}KG}=0.13 s⁻¹, T₁(Glu C2)=2.5 s (calculated based on data from this study); kα_{KG→Glu}≈9.6 s⁻¹ (assuming kα_{KG→Glu}[α-KG]=k_{Glu→\tilde{α}KG}[Glu])>>T₁⁻¹(α-KG), T₂⁻¹(α-KG); T₂(Glu C2)≈0.2 s (estimated from ref (Nirmala et al., “Measurement of ¹³C spin-spin relaxation times by two-dimensional heteronuclear ¹H-¹³C correlation spectroscopy,” J Magn Reson 82:659-661, 1989). Using Eq. [32] in Zhou et al., “Quantitative description of proton exchange processes between water and endogenous and exogenous agents for WEX, CEST, and APT experiments,” Magn Reson Med. 51:945-952, 2004, γBₗₛₐₜ>36 Hz is required for a <5% saturation transfer error in (ΔM/M₀)_{Glu}. The large chemical shift dispersion (>18000 Hz at 11.7 T) between the α-carbons of amino acids and the carbonyl carbons of their cognate α-keto acids provides an ideal situation for saturation transfer experiments without significant interference from the “RF spillover” effects between the saturated spins and the observed spins. The lowest γBₗₛₐₜ field strength used in this study for saturating α-KG was 158 Hz, which is far greater than required for its complete saturation. At γBₗₛₐₜ=158 Hz, the estimated saturation transfer error in (ΔM/M₀)_{Glu} is less than 0.3%. The T₁ of Glu C2 is only ˜70% longer than the metabolite T₁ measured from the proton spectra at the same field strength (Shen et al., “A new strategy for in vivo spectral editing. Application to GABA editing using selective homonuclear polarization transfer spectroscopy,” J Magn Reson. 170:290-298, 2004), confirming previous description of relatively short ¹³C T₁'s observed in vivo (Alger et al., “NMR studies of enzymatic rates in vitro and in vivo by magnetization transfer,” Q Rev Biophys. 17:83-124, 1984). The relatively small γBₗₛₐₜ required for complete saturation of α-KG and the relatively short ¹³C T₁ measured in vivo indicates that the metabolic fluxes of AST could be detectable using the more sensitive proton magnetic resonance spectroscopy methods. It is also possible to use the ¹³C→¹H heteronuclear Hartmann-Hahn transfer or polarization transfer (e.g., INEPT or DEPT) techniques for in vivo proton detection of the CMT effects.

The concentration of OAA in vivo has been estimated to be 3.6-5.7 μmol/kg (Duffy et al., “Cerebral carbohydrate metabolism during acute hypoxia and recovery,” J Neurochem. 19:959-977, 1972; Siesjo, Brain energy metabolism, John Wiley & Sons, Chichester, 1978), which is more dilute than any other tricarboxylic acid cycle intermediates. Free OAA is chemically unstable. It readily decarboxylates into pyruvate and bicarbonate at neutral pH, particularly at the presence of metal cations. OAA is a substrate for AST, citrate synthase, and malate dehydrogenase. The concentration of some of these enzymes is similar to or higher than that of OAA. Since an appreciable amount of a substrate will be enzyme-bound when enzymes are present in high concentrations, a significant portion of OAA may be bound to enzymes. In particular, binding to citrate synthase has been shown to render chemical stability to OAA and cause a large shift of its carbonyl carbon resonant frequency (Kurz et al., “Evidence from ¹³C NMR for polarization of the carbonyl of oxaloacetate in the active site of citrate synthase,” Biochemistry 24:452-457, 1985). The combined effect of its tiny pool size (i.e., short apparent T₂) and its binding to proteins renders complete RF saturation of OAA more difficult than α-ketoglutarate. Assuming that OAA is fully saturated with γBₗₛₐₜ=790 Hz and the T₁ of Asp C2 in the absence of exchange is the same as that of Glu C2, k_{Asp→OAA} is estimated to be ˜0.15 s⁻¹. The corresponding V_{Asp→OAA} is estimated to be ˜26 μmol/g/min.

### EXAMPLE 2

In this example, the in vivo CMT effect was further validated in rhesus monkey brain at 4.7 Tesla. In particular, the AST CMT effect was observed for the first time in the carboxylic carbon region by saturating the carboxylic carbon (Cl) of α-KG.

Method

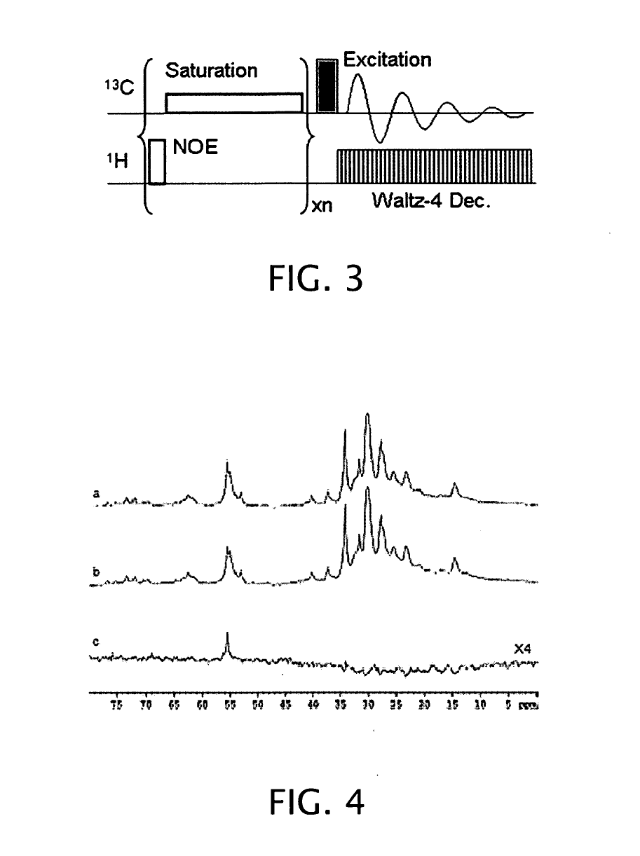

The experiments were performed on a Bruker spectrometer interfaced to a 4.7T 30-cm bore horizontal magnet. A 13C surface coil (Dia.=6 cm) was placed inside a double-D linear 1H coil formed on the surface of a 12-cm diameter cylindrical tube (Li et al., ISMRM Proc. p 346:4, 2005, Li et al., NMR Biomed, 2005, in press). Three female rhesus monkeys (5˜6 kg) were placed in prone position and anesthetized with intravenous infusion of propofol (0.4 mg/kg/min) and intramuscular injection of dormitor (0.02 mg/kg). Administration of a solution of 20% w/w 99%-enriched [1-¹³C]glucose began with a 10˜12 ml bolus injection followed by continuous intravenous infusion to maintain glucose concentration at 20˜25 mM. The pulse sequence is shown in FIG. 3. This sequence was used for both C1 and C2 acquisitions. For the C2 spectra, a 1-ms adiabatic half-passage pulse (in gray) was used for the ¹³C excitation. TR=10000 ms. Saturation of the carbonyl carbon of α-KG at 206.0 ppm was performed by applying a train of 200 ms rectangular pulses with nominal γBₗₛₐₜ=250 Hz interrupted by a train of hard nominal 180° proton pulses for generation of NOE. The sequence was interleaved every FID with each saturation transfer spectrum followed by a control spectrum. When control spectra were acquired, the saturation pulse was placed at an equal spectral distance from Glu C2 but on the opposite side of the α-KG C2. For the C1 spectra, a nominal 30° 50 μs hard pulse was used for ¹³C excitation. TR=1500 ms. The same saturation/NOE pulse train was used except that nominal γBₗₛₐₜ=30 Hz. Blocks of saturation and control spectra were interleaved (256 FIDs per block). For observing CMT effect in the carboxylic carbon region, the C1 carbon of α-KG at 170.3 ppm was saturated. When the control spectra were acquired, the saturation pulse was turned off. In both C1 and C2 experiments, proton decoupling was achieved by the use of the WALTZ-4 scheme with its nominal 90° pulse set to 400 μs.

Results and Discussion

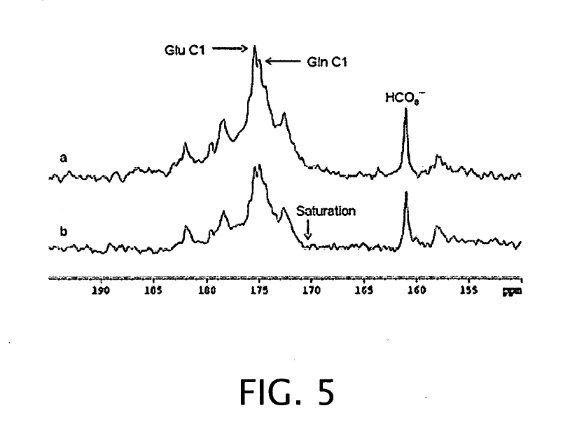

FIG. 4 shows a comparison of a saturation transfer spectrum with the corresponding control spectrum for the Glu→α-KG reaction when the carbonyl carbon of α-KG at 206.0 ppm was saturated. Spectrum 4a is the control spectrum. The corresponding saturation transfer spectrum is shown in FIG. 4b. FIG. 4c shows the difference spectrum. A total of 512 interleaved scans were accumulated. Data acquisition was initiated 90 minutes after the start of ¹³C glucose infusion. 10 Hz exponential line broadening was applied before Fourier transform. A significant intensity change in Glu C2 at 55.5 ppm was observed as expected. The results for the carboxylic carbon region are shown in FIG. 5, where 5a is the control spectrum without any pre-irradiation and 5b is the saturation transfer spectrum. Data acquisition was initiated two hours after the start of ¹³C-glucose infusion. 10 Hz exponential line broadening was applied before Fourier transform. A total of 8 interleaved data blocks (total NS=2048) were accumulated. To compensate for the distortion caused by the nearby saturation pulse at 170.3 ppm, different phase and baseline corrections were made to the two spectra. An overall reduction in signal intensity in FIG. 5b was observed, which is, at least partially, due to the close proximity between the saturation pulse (170.3 ppm) and the carboxylic carbons (˜175 ppm). In the control spectrum (5a), Glu C1 is higher than Gln C1. In the saturation transfer spectrum (5b), Glu C1 is lower than Gln C1 although Glu C1 at 175.3 ppm lies farther from the saturation pulse than Gln C1 at 174.8 ppm, demonstrating significant saturation transfer effect caused by the rapid exchange between α-KG and Glu although a much weaker saturation pulse (nominal γBₗₛₐₜ=30 Hz) was used here. Considering previous observation of relatively short 13C T1 in vivo, the large magnetization transfer effect observed in FIGS. 4 and 5 indicates a very fast exchange between α-KG and Glu in the anesthetized rhesus monkey brain, consistent with our previous observation in anesthetized rat brain where the unidirectional Glu→α-KG flux has been quantified to be 78 μmol/g/min. The results shown in FIGS. 4 and 5, as well as those from our previous rat studies unambiguously demonstrate the existence of a fast exchange between α-KG and Glu in vivo in brain, fast enough to support a rapid exchange between α-KG and Glu across mitochondrial inner membrane.

In view of the many possible embodiments to which the principles of the disclosed methods may be applied, it should be recognized that the illustrated embodiments are only preferred examples and should not be taken as limiting the scope of the invention. Rather, the scope of the invention is defined by the following claims. We therefore claim as our invention all that comes within the scope and spirit of these claims.

We claim:

1. A non-invasive method of assessing aspartate aminotransferase (AST) activity in vivo, comprising performing in vivo magnetization transfer spectroscopy on an organ of a living subject in which AST activity is to be measured, and determining a change in magnetic resonance signal intensity of a reactant in an AST catalyzed reaction.

2. The method of claim 1, wherein the AST catalyzed reaction is the interconversion of aspartate (ASP) and α-ketoglutarate (α-KG) with glutamate (Glu) and oxaloacetate (OAA).

3. The method of claim 1, wherein performing magnetization transfer spectroscopy comprises obtaining at least two carbon-13 magnetic resonance spectra and calculating a rate of the AST catalyzed reaction.

4. The method of claim 1, wherein performing magnetization transfer spectroscopy further comprises:
obtaining a first carbon-13 magnetic resonance spectrum with signal enhancement by the nuclear Overhauser effect and proton decoupling;
obtaining a second carbon-13 magnetic resonance spectrum with simultaneous signal enhancement by the nuclear Overhauser effect and proton decoupling and saturation of the carbonyl magnetic resonance of α-ketoglutarate or oxaloacetate; and
obtaining a difference spectrum to reveal the magnetization transfer effect, wherein the difference spectrum indicates the AST activity.

5. The method of claim 4, further comprising measuring Tₗₙᵤₗₗ of metabolites with simultaneous saturation of corresponding carbonyl signals.

6. The method of claim 3, wherein a carbonyl carbon or carboxylic carbon of α-ketoglutarate or oxaloacetate is selectively saturated.

7. The method of claim 1, wherein the method is organ-selective.

8. The method of any claim 1, wherein the organ is the brain.

9. The method of claim 4, wherein the saturation of the carbonyl magnetic resonance of α-ketoglutarate or oxaloacetate is placed at an equal spectral distance but on the opposite side of the carbonyl resonance frequency of α-ketoglutarate or oxaloacetate.

10. The method of claim 3, wherein the carbon-13 magnetic resonance spectra are obtained via direct ¹³C-detected magnetic resonance spectroscopy, proton detection of carbon to proton coherence transfer, or direct proton detection.

11. The method of claim 4, wherein the nuclear Overhauser effect is measured between an α-proton of glutamate and an α-carbon of glutamate.

12. The method of claim 11, wherein the proton decoupling includes the decoupling of protons attached to glutamate.

13. The method of claim 4, wherein the nuclear Overhauser effect is generated prior to generation of the proton decoupling.

14. The method of claim 4, wherein obtaining the second carbon-13 magnetic resonance spectrum includes simultaneously executing a saturation pulse on the carbonyl resonance frequency of α-ketoglutarate or oxaloacetate and a nuclear Overhauser effect pulse on glutamate or aspartate.

15. The method of claim 1, further comprising administering to the subject a ¹³C isotope.

16. A method for detecting a physiological or pathological condition of an organ of a living subject, wherein a change in aspartate aminotransferase (AST) activity in the organ is indicative of the presence of a physiological or pathological condition, the method comprising:
performing in vivo magnetization transfer spectroscopy on an organ of a living subject in which AST activity is to be measured and determining a change in magnetic resonance signal intensity of a reactant in an AST catalyzed reaction; and
determining whether the measured AST activity indicates the presence of a physiological or pathological condition.

17. The method of claim 16, wherein the pathological condition is a neurological disease.

18. The method of claim 17, the neurological disease is Huntington's disease, olivopontocerebellar atrophy, epilepsy, and schizophrenia.

19. The method of claim 16, wherein the pathological condition is hepatitis, cholangitis, Gilbert's disease, cirrhosis, cardiac infarction, muscle dystrophy, leukemia, kidney inflammation, or the presence of a tumor.

20. The method of claim 16, wherein the AST catalyzed reaction is the interconversion of aspartate (ASP) and α-ketoglutarate (α-KG) with glutamate (Glu) and oxaloacetate (OAA).

21. The method of claim 16, wherein performing magnetization transfer spectroscopy further comprises:
obtaining a first carbon-13 magnetic resonance spectrum with signal enhancement by the nuclear Overhauser effect and proton decoupling;
obtaining a second carbon-13 magnetic resonance spectrum with simultaneous signal enhancement by the nuclear Overhauser effect and proton decoupling and saturation of the carbonyl magnetic resonance of α-ketoglutarate or oxaloacetate; and
obtaining a difference spectrum to reveal the magnetization transfer effect, wherein the difference spectrum indicates the AST activity.

22. The method of claim 21, wherein the saturation of the carbonyl magnetic resonance of α-ketoglutarate or oxaloacetate is placed at an equal spectral distance but on the opposite side of the carbonyl resonance frequency of α-ketoglutarate or oxaloacetate.

23. The method of claim 21, wherein the nuclear Overhauser effect is measured between an α-proton of glutamate and an α-carbon of glutamate.

24. The method of claim 21, wherein obtaining the second carbon-13 magnetic resonance spectrum includes simultaneously executing a saturation pulse on the carbonyl resonance frequency of α-ketoglutarate or oxaloacetate and a nuclear Overhauser effect pulse on glutamate or aspartate.

25. The method of claim 16, further comprising administering to the subject a ¹³C isotope.
